(19) United States
(12) Patent Application Publication
(10) Pub. No.: US 2015/0225339 A1
(43) Pub. Date: Aug. 13, 2015

(54) FLUORINATED POLYMERIZABLE COMPOUND

(71) Applicant: BASF SE, Ludwigshafen (DE)

(72) Inventors: Fabian Niedermair, Trostberg (DE); Norbert Steidl, Kienberg (DE); Burkhard Walther, Garching (DE); Franz Wolfertstetter, Palling (DE)

(21) Appl. No.: 14/423,558

(22) PCT Filed: Oct. 4, 2013
(86) PCT No.: PCT/EP2013/070711

§ 371 Date: Feb. 24, 2015

(30) Foreign Application Priority Data
Oct. 4, 2012 (EP) 12187200.6

(51) Int. Cl. C07C 275/18; C08F 122/10; C09D 133/16; C07C 273/02

(52) U.S. Cl. CPC C07C 275/18; C07C 273/02; C08F 122/10; C09D 133/16

(57) ABSTRACT

The invention relates to a polymerizable compound of formula (I), where R_{f} represents a monovalent fluorinated organic radical of 1 to 18 carbon atoms and 35 to 85 wt % of fluorine, R¹ represents a moiety from the group consisting of cyclic or acyclic alkylene of 5 to 14 carbon atoms, arylene, diarylenealkane and dialkylene-substituted aryl, B represents —O— or —N(R²)—, R² represents hydrogen, an aliphatic hydrocarbyl moiety of 1 to 12 carbon atoms or a monovalent fluorinated organic radical of 1 to 18 carbon atoms and 35 to 85 wt % of fluorine, X represents a polymeric polyol having two or more repeating units and after removal of two OH groups, E represents —O— or —N(R³)—, R³ represents a hydrocarbyl moiety of 1 to 12 carbon atoms or a hydrogen atom, Y represents a chemical bond or a divalent moiety of 2 to 20 carbon atoms, and Z represents an organic moiety comprising a polymerizable building-block group. The invention further discloses a process for forming this compound, polymers comprising the polymerizable compound of the invention and the use of said polymers for coating a surface.

The present invention relates to fluorinated polymerizable compounds. The invention further discloses a process for forming this compound, polymers comprising the polymerizable compound of the present invention and its use for coating surfaces.

Coating compositions based on fluorinated polymers have long been known to be useful for many different purposes.

EP-B-856 020, for instance, describes nonaqueous clear coating compositions based on fluorinated polyacrylate binders and polyisocyanate crosslinking agents, which can be cured to form oil-, dirt- and water-repellent finishes and which can be used for coating vehicles, such as subways, trains, buses and the like, industrial equipment, for example tanks, buildings and other structures. The clear coating compositions are particularly useful for coating buildings, buses, trains and other objects where graffiti is a problem, since the resulting finishes are easy to clean and also offer good resistance to dirt. The fluorinated polyacrylate binders are prepared using ethylenically unsaturated compounds having fluoroalkyl groups, for example fluoroalkylethyl (meth)acrylates or 2-(N-ethylperfluorooctanesulfoamido)ethyl methacrylate. However, these fluorinated monomers have to be added on their own and separately from the other monomers, so the binders are fairly inconvenient to synthesize.

EP-B-856 022 discloses similar coating compositions which, in addition to fluorinated polyacrylate binders, comprise, as crosslinkers, polyisocyanates where from 0.1 to 33 mol % of isocyanate groups have been reacted with perfluoroalcohols. The method described therein of forming the fluorinated polyisocyanates is likely to produce not only monofluorinated polyisocyanates but also high proportions of di- and trifluorinated polyisocyanates which can migrate to the finish surface and thus have an adverse effect on the appearance, the hardness, the chemical resistance and other physical properties of the finish.

Fluorinated clear coating compositions are also described in WO05/030892, wherein the binders in these clear coating compositions comprise fluorinated polyacrylates which additionally comprise polymerized organosilane monomers. The fluorinated polyacrylates are in turn obtained by incorporating polymerized units of ethylenically unsaturated compounds having fluoroalkyl groups, for example fluoroalkylethyl (meth)acrylates or 2-(N-ethylperfluorooctanesulfoamido)alkyl (meth)acrylate.

WO05/030891 discloses fluorinated clear coating compositions having improved clear coat/clear coat adherence which, in addition to a fluorinated silane polymer based on polymerized units of ethylenically unsaturated compounds having fluoroalkyl groups, for example fluoroalkylethyl (meth)acrylates or 2-(N-ethylperfluorooctanesulfoamido)-alkyl (meth)acrylates, comprise a fluorinated polyurethane resin for improved adherence. This resin is obtainable by reacting a polyisocyanate with a perfluorinated monoalcohol and an oligomeric and/or polymeric polyether polyol, such as ethoxylated-propoxylated glycol, and has no free isocyanate groups left.

WO05/080465 describes easy-to-clean, abrasion-resistant and alkali-resistant coats, which are obtained by using coating compositions which in addition to a curable binder system and inorganic particles comprise at least one fluorinated polymer or oligomer having at least one functional group capable of reacting with a functional group of the binder system. The fluorinated polymers or oligomers used include for example fluorinated polyethers, fluorinated epoxides, fluorinated polyurethanes and fluorinated chain growth addition polymers prepared using commercially available fluoromonomers, such as tetrafluoroethylene, vinylidene fluoride, and the like.

WO07/115752 describes two-component aqueous hybrid reactive resin systems based on polyurethane and having an epoxy/amine curing mechanism, which are used in the building and industrial sectors for forming mechanically strong easy-to-clean coatings. These coating compositions may optionally comprise amino- and/or hydroxyl- and/or mercapto-functional fluoromodified macromonomers or telechelics.

WO05/007762 describes aqueous, optionally fluorinated polyurethane hybrid dispersions having covalently attached fluorinated side chains which can be introduced via the polyurethane base and/or via free-radically polymerizable fluorinated monomers. These polyurethane hybrid dispersions combine high crosslink density with high hardness and so can be used to form dirt-repellent coatings having good mechanical properties and good solvent and chemical resistance, which are useful for many different purposes.

WO08/040428 discloses fluoromodified polyurethane coatings especially in the building and industrial sectors for permanent oil-, water- and dirt-repellent coating of mineral and nonmineral substrates.

EP-A-2 085 442 discloses aqueous coating compositions based on fluorosilane components, which are used for permanent oil- and water-repellent surface treatment of mineral and nonmineral substrates for various applications.

EP-B-587 667 discloses coating compositions based on fluorinated inorganic polycondensates, which are used for coating of glass, ceramic, metal, plastics and paper, especially for coating of exterior and interior mirrors and also windscreens of motor vehicles.

EP-A-1 844 863 likewise describes coating compositions for forming strongly liquid-repellent finishes, i.e., finishes on which a reference liquid, especially water, has a very large contact angle of 120° to 180°. The coating compositions used for this comprise a polymer and ceramic materials/nanoparticles and thus lead to finishes having a textured surface. No particulars are provided regarding the polymers' exact composition.

WO96/04123 describes hydrophobic coating compositions for forming self-cleaning surfaces having an artificial texture made up of depressions and elevations where the elevations consist of hydrophobized materials. Teflon is used for example.

Comb-shaped polymers having fluorinated side chains have already been tried for surface coating. It is known in this respect that very long fluorinated chains and very short spacers from the main chain, in the form of hydrocarbyl groups, are decisive if low surface energy and hence good dirt- and water-repellent properties are to be achieved. Comparatively long spacers lead to distinctly worse results (“Fluorinated comblike homopolymers: The effect of spacer lengths on surface properties”, Saïdi, Salima; Guittard, Frédéric; Guimon, Claude; Géribaldi, Serge; Journal of Polymer Science Part A: Polymer Chemistry, vol. 43, issue 17, pp. 3737-3747).

The problem addressed by the present invention was therefore that of further improving existing coating compositions that are based on fluorinated polymers. The problem was more particularly that of providing fluorinated monomers which even when used at low levels and when having a comparatively low fluorine content endow the resulting finishes with outstanding water- and dirt-repellent properties.

The problem was further that of providing fluorinated monomers which, in the products formed therefrom, ensure very good mechanical and photochemical stability, good appearance, a very high hardness and good chemical resistance. The problem was also that of ensuring that the fluorinated monomers are obtainable in a very simple and economical manner.

The problem was solved by a polymerizable compound of formula (I),

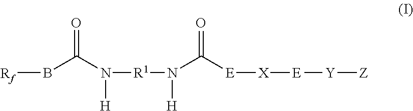

where
R_{f} represents a monovalent fluorinated organic radical of 1 to 18 carbon atoms and 35 to 85 wt % of fluorine,
R¹ represents a moiety from the group consisting of cyclic or acyclic alkylene of 5 to 14 carbon atoms, arylene, diarylenealkane and dialkylene-substituted aryl,
B represents —O— or —N(R²)—
R² represents hydrogen, an aliphatic hydrocarbyl moiety of 1 to 12 carbon atoms or a monovalent fluorinated organic radical of 1 to 18 carbon atoms and 35 to 85 wt % of fluorine,
X represents a polymeric polyol having two or more repeating units and after removal of two OH groups
E represents —O— or —N(R³)—,
R³ represents a hydrocarbyl moiety of 1 to 12 carbon atoms or a hydrogen atom,
Y represents a chemical bond or a divalent moiety of 2 to 20 carbon atoms, and
Z represents an organic moiety comprising a polymerizable building-block group.

Surprisingly, not only is the stated problem fully solved, but the monomers are also simple to convert into polymers. Moreover, no solvent is needed at all to form the monomers of the present invention.

A particular surprise in view of the prior art was that very good dirt- and water-repellent properties are obtained for the polymers formed therefrom notwithstanding the presence of a spacer between the fluorinated organic moiety R_{f} and the polymerizable building-block group Z.

It is particularly preferable for the group X to represent a polymeric polyol having two or more repeating units and after removal of two OH groups, selected from the group consisting of polyesters, polyalkylene ethers, polycarbonates, polyester carbonates, polyacetals, polyurethane polyols and polysiloxane polyol. Preferred polymeric polyols include polyesters, polyalkylene ethers, polyester carbonates and polycarbonates.

Suitable polymeric polyols after removal of two OH groups are preferably in the molecular weight range from 72 to 3000, preferably from 100 to 2000 and more preferably from 150 to 1000.

The polyalkylene ethers of the present invention can be represented by the general structural formula (II), in which case each R⁴ may be the same or different and n represents an integer from 1 to 70, preferably from 2 to 40 and especially from 2 to 20.

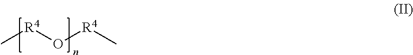

Polyalkylene glycols such as polyethylene glycols, polypropylene glycols and polyepichlorohydrins as well as epoxy resins, polytetrahydrofurans, polyoxetanes, polyphenylene ethers or polyether ketones for example may be concerned according to the present invention. Polyalkylene glycols are predominantly linear polyethers, i.e., polymers having terminal hydroxyl groups. Preferred representatives of these polyether polyols for the purposes of the present invention include the polyethylene glycols, polypropylene glycols and polytetramethylene glycols obtained by polyaddition, especially KOH- or DMC-catalyzed polyaddition, of respectively ethylene oxide, propylene oxide and tetrahydrofuran onto water. Preference is further given to block copolymers formed from ethylene oxide and propylene oxide.

More particularly, the polyalkylene ether may be a building-block group of formula (III)

—(CₘH₂ₘO)ₙ—(CₘH₂ₘ)— (III)

where each m represents independently for every (CₘH₂ₘO) unit an integer between 2 and 18 and is the same or different and a represents an integer between 1 and 30.

Of the polyalkylene ethers which can be employed according to the present invention, those comprising C₅-C₁₀₀ alkylene groups can be prepared via polycondensation reactions from diols in the presence of sulfosuccinic acid as catalyst.

Useful diol components include, in particular, saturated or unsaturated, branched or unbranched aliphatic dihydroxy compounds of 5 to 36 carbon atoms or aromatic dihydroxy compounds, for example 1,5-pentanediol, 1,6-hexanediol, neopentylglycol, bis(hydroxymethyl)cyclohexanes, bisphenol A, dimer diols, hydrogenated dimer diols or else mixtures thereof.

The polymeric polyol of the present invention may comprise polyalkylene ethers obtained by polycondensation of a dimer diol or of a C₅-C₃₆ α,ω-alkanediol. The polycondensation is preferably carried out in the presence of sulfosuccinic acid, but can similarly be carried out with other catalysts having the same effect. Dimer diols are mixtures because of their method of production; their method of production is well-known from the prior art, for example from DE 1 768 313 and U.S. Pat. No. 2,347,562. Preferred dimer diol components for conversion into polyalkylene ethers useful for the purposes of the present invention are dimer diols having a carbon atom total of C₁₂-C₁₀₀. Of particular suitability are C₁₂-C₄₀ dimer diols, preferably C₁₂-C₂₀ and more preferably C₁₂-C₁₆ dimer diols, the stated carbon chain length being based on one chain.

It is particularly preferable for the group X to represent a polymeric lactone-based polyol having two or more repeating units and after removal of two OH groups. What may be concerned here is a homo- or copolymer of a lactone, preferably a hydroxyl-terminated addition product of a lactone on a suitable difunctional starter molecule. Examples of suitable lactones are [ε]-caprolactone, [β]-propiolactone, [γ]-butyrolactone and/or methyl-[ε]-caprolactone, and also mixtures thereof. Instead of chain growth addition polymers of lactones, especially the corresponding chemically equivalent polycondensates of the hydroxy carboxylic acids corresponding to the lactones can also be used with preference.

In one preferred embodiment, therefore, the group X may be a polyester. More particularly, the polyester may be a building-block group of formula (IV)

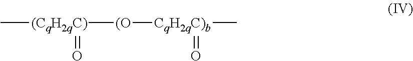

where each q represents independently for every (C_{q}H_{2q}(C═O)) unit an integer between 2 and 18 and is the same or different and b represents an integer between 1 and 30.

The group X may further represent a polymeric polyol, after removal of two OH groups with two or more repeating units, selected from the group of hydroxyl-containing polycarbonates. Compounds contemplated here are obtainable by reaction of carbonic acid derivatives, for example diphenyl carbonate, dimethyl carbonate or phosgene, with diols. Particularly useful diols include for example ethylene glycol, 1,2-propanediol, 1,3-propanediol, 1,3-butanediol, 1,4-butanediol, 1,6-hexanediol, 1,8-octanediol, neopentylglycol, 1,4-bishydroxymethylcyclohexane, 2-methyl-1,3-propanediol, 2,2,4-trimethylpentanediol, 1,3-dipropylene glycol, polypropylene glycols, dibutylene glycol, polybutylene glycols, bisphenol A and tetrabromobisphenol A.

It is essential for the purposes of the present invention that R¹ in formula (I) represent a moiety from the group consisting of cyclic or acyclic alkylene of 5 to 14 carbon atoms, arylene, diarylenealkane and dialkylene-substituted aryl. In one preferred embodiment, R¹ is selected from the group consisting of methylene, ethylene, propylene, butylene, pentamethylene, hexamethylene, octamethylene, 2-ethylhexamethylene, phenylene, tolylene, methylenebisphenyl, propylenebisphenyl, cyclohexylene, methylene- or propylenebiscyclohexyl moieties and a group obtained by removing the isocyanate groups from 1-isocyanato-3,3,5-trimethyl-5-isocyanatomethylcyclohexane. It is particularly preferable for R¹ to be a group obtained by removing the isocyanate groups from 1-isocyanato-3,3,5-trimethyl-5-isocyanatomethylcyclohexane.

The group Y represents a chemical bond or a divalent moiety of 2 to 20 carbon atoms. When Y is not a chemical bond, Y is preferably selected from the group consisting of methylene, ethylene, propylene, butylene, pentamethylene, hexamethylene, octamethylene, 2-ethylhexamethylene, phenylene, tolylene, methylenebisphenyl, propylenebisphenyl, cyclohexylene, methylene- or propylenebiscyclohexyl moieties and a group obtained by removing the isocyanate groups from 1-isocyanato-3,3,5-trimethyl-5-isocyanatomethylcyclohexane. It is particularly preferable for Y to be a group obtained by removing the isocyanate groups from 1-isocyanato-3,3,5-trimethyl-5-isocyanatomethylcyclohexane.

It is preferable for R_{f} to be a group obtained by removing the hydroxyl group from at least one of the following compounds:

3,3,4,4,5,5,6,6,7,7,8,8,8-tridecafluorooctan-1-ol, 3,3,4,4,5,5,6,6,7,7,8,8,9,9,10,10,10-heptadecafluorodecan-1-ol, 3,3,4,4,5,5,6,6,7,7,8,8,9,9,10,10,11,11,12,12,12-heneicosafluorododecan-1-ol, 3,3,4,4,5,5,6,6,7,7,8,8,9,9,10,10,11,11,12,12,13,13,14,14,14-pentacosafluorotetradecan-1-ol, 3,3,4,4,5,5,6,6,7,7,8,8,9,9,10,10,11,11,12,12,13,13,14,14,15,15,16,16,16-nonacosafluorohexadecan-1-ol, 3,3,4,4,5,5,6,6,7,7,8,8-dodecafluoroheptan-1-ol, 3,3,4,4,5,5,6,6,7,7,8,8,9,9,10,10-hexadecafluorononan-1-ol, 3,3,4,4,5,5,6,6,7,7,8,8,9,9,10,10,11,11,12,12-eicosafluoroundecan-1-ol, 3,3,4,4,5,5,6,6,7,7,8,8,9,9,10,10,11,11,12,12,13,13,14,14-tetracosafluorotridecan-1-ol, 3,3,4,4,5,5,6,6,7,7,8,8,9,9,10,10,11,11,12,12,13,13,14,14,15,15,16,16-octacosafluoropentadecan-1-ol, 4-(3,3,4,4,5,5,6,6,7,7,8,8,8-tridecafluorooctyl)benzyl alcohol, 4-(3,3,4,4,5,5,6,6,7,7,8,8,9,9,10,10,10-heptadecafluorodecyl)benzyl alcohol, 4-(4,4,5,5,6,6,7,7,8,8,9,9,9-tridecafluorononyloxyl)benzyl alcohol, 4-(4,4,5,5,6,6,7,7,8,8,9,9,10,10,11,11,11-heptadecafluoroundecyloxy)benzyl alcohol and also the commercial products Fluowet® EA 600, Fluowet® EA 800, Fluowet® EA 093, Fluowet® EA 612, Fluowet® EA 612 N, Fluowet® EA 812 AC, Fluowet® EA 812 IW, Fluowet® EA 812 EP, Fluowet® EA 6/1020, Fluowet® PA, consisting of perfluoroalkylethanol mixtures, Fluowet® OTL, Fluowet® OTN, consisting of ethoxylated perfluoroalkylethanol mixtures, from Clariant GmbH, the commercial products A-1620, A-1630, A-1660, A-1820, A-1830, A-1860, A-2020, A-3620, A-3820, A-5610, A-5810 from Daikin Industries, Ltd., the commercial products Zonyl® BA, Zonyl® BA L, Zonyl® BA LD, Foralkyl® EOH-6N LW, consisting of perfluoroalkylethanol mixtures, Zonyl® OTL, Zonyl® OTN, consisting of ethoxylated perfluoroalkylethanol mixtures, Zonyl® FSH, Zonyl® FSO, Zonyl® FSN, Zonyl® FS-300, Zonyl® FSN-100, Zonyl® FSO-100 from Du Pont de Nemours, the commercial products Krytox® from Du Pont de Nemours, consisting of hexafluoropropene oxide (HFPO) oligomer-alcohol mixtures, or suitable combinations thereof. Preferably perfluoroalkylethanol mixtures with 30-49.9 wt % of 3,3,4,4,5,5,6,6,7,7,8,8,8-tridecafluorooctan-1-ol and 30-49.9 wt % of 3,3,4,4,5,5,6,6,7,7,8,8,9,9,10,10,10-heptadecafluorodecan-1-ol such as the commercial products Fluowet® EA 612 and Fluowet® EA 812. The commercial products A-1620, A-1820 from Daikin Industries, Ltd. are also possible.

It is further preferable to introduce the R_{f} moiety by using an amine, especially R_{f}NH₂. R_{f} is therefore preferably a group obtained by removing the amino group from at least one of the following compounds:

3,3,4,4,5,5,6,6,7,7,8,8,8-tridecafluorooctylamine, 3,3,4,4,5,5,6,6,7,7,8,8,9,9,10,10,10-heptadecafluorodecylamine, 3,3,4,4,5,5,6,6,7,7,8,8,9,9,10,10,11,11,12,12,12-heneicosafluorododecylamine, 3,3,4,4,5,5,6,6,7,7,8,8,9,9,10,10,11,11,12,12,13,13,14,14,14-pentacosafluorotetradecylamine, 3,3,4,4,5,5,6,6,7,7,8,8,9,9,10,10,11,11,12,12,13,13,14,14,15,15,16,16,16-nonacosafluorohexadecylamine, 4-(3,3,4,4,5,5,6,6,7,7,8,8,8-tridecafluorooctyl)-benzylamine, 4-(3,3,4,4,5,5,6,6,7,7,8,8,9,9,10,10,10-heptadecafluorodecyl)-benzylamine, and also 1,1,1,2,2,3,3,4,4,5,5,6,6-tridecafluoro-8-iodooctane, 1,1,1-2,2,3,3,4,4,5,5,6,6,7,7,8,8-heptadecafluoro-10-iododecane, 1,1,1,2,2,3,3,4,4,5,5,6,6,7,7,8,8,9,9,10,10-heneicosafluoro-12-iodododecane, 1,1,1,2,2,3,3,4,4,5,5,6,6,7,7,8,8,9,9,10,10,11,11,12,12-pentacosafluoro-14-iodotetradecane, 1,1,1,2,2,3,3,4,4,5,5,6,6,7,7,8,8,9,9,10,10,11,11,12,12,13,13,14,14-nonacosafluoro-16-iodohexadecane, the commercial products Fluowet® I 600, Fluowet® I 800, Fluowet® I 612, Fluowet® I 812, Fluowet® I 6/1020, Fluowet® I 1020, consisting of perfluoroalkyl iodide mixtures, Fluowet® EI 600, Fluowet® EI 800, Fluowet® EI 812, Fluowet® EI 6/1020, consisting of perfluoroalkylethyl iodide mixtures, from Clariant GmbH and suitable amination reagents, the commercial products U-1610, U-1710, U-1810 from Daikin Industries, Ltd. or suitable combinations thereof. Preference is given to using perfluoroalkylethanol mixtures with 30-49.9 wt % of 3,3,4,4,5,5,6,6,7,7,8,8,8-tridecafluorooctylamine and 30-49.9 wt % of 3,3,4,4,5,5,6,6,7,7,8,8,9,9,10,10,10-heptadecafluorodecylamine.

In one particularly preferred embodiment, R_{f} is at least one moiety of general formula (V),

CF₃—(CF₂)_{z}—(CH₂)_{y}— (V)

where each z represents an integer from 0 to 16 and is the same or different, and each y represents an integer from 1 to 6 and is the same or different.

It is particularly preferable for R_{f} to be a moiety of the formula CF₃—(CF₂)₅—(CH₂)₂—.

Z may comprise a multiplicity of different organic moieties as long as they meet the prerequisite of comprising a polymerizable building-block group.

It is preferable for Z to be at least one moiety from the group consisting of acrylate, methacrylate, oxyalkylacrylate, oxyalkylmethacrylate, dialkanolaminyl, oxyalkylcaprolactoneacrylate, oxyalkylcaprolactonemethacrylate, oxycycloalkylmethacrylate, oxycycloalkylacrylate and oxyalkylmaleimide, preferably oxyalkylmethacrylate, dialkanolaminyl and oxyalkylcaprolactonemethacrylate.

In one particularly preferred embodiment, the polymerizable compound of formula (I) is a compound from the group consisting of (VI), (VII), (VIII) and (IX), which are represented by the following formula:

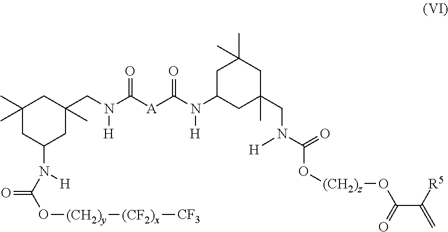

where
A represents a building-block group of the formula —O—(CₘH₂ₘO)ₐ— where each m represents independently for every (CₘH₂ₘO) unit an integer from 2 to 4 and is the same or different and a represents an integer from 2 to 30,
R⁵ represents hydrogen or methyl,
z represents an integer from 1 to 5,
x represents an integer from 0 to 9,
y represents an integer from 2 to 12,

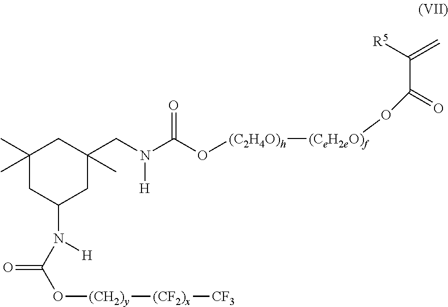

where
R⁵ represents hydrogen or methyl,
e in each occurrence represents 2 or 3 independently for every (CₑH₂ₑO) unit and is the same or different,
f represents an integer from 0 to 150,
h represents an integer from 0 to 50 subject to the proviso that the sum total of f and h is not less than 3,
x represents an integer from 0 to 9,
y represents an integer from 2 to 12,

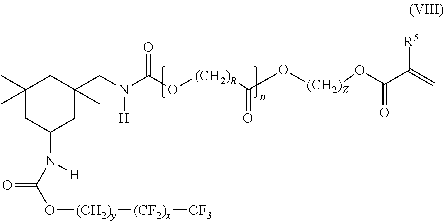

where
Z represents an integer from 1 to 50, in particular 1 to 5,
R represents an integer from 2 to 18, in particular 2 to 8,
n represents an integer from 1 to 10,
x represents an integer from 0 to 9,
y represents an integer from 2 to 12,

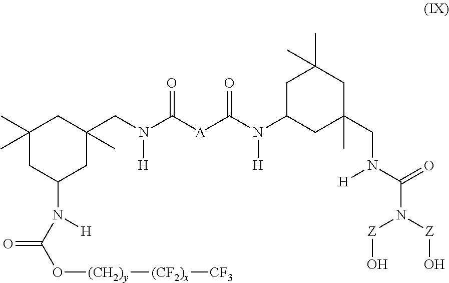

where
A represents a building-block group of the formula —O—(CₘH₂ₘO)ₐ— where each m represents independently for every (CₘH₂ₘO) unit an integer from 2 to 4 and is the same or different and a represents an integer from 2 to 30,
Z represents a (CₚH₂ₚ) unit,
p represents an integer from 2 to 8,
x represents an integer from 0 to 9,
y represents an integer from 2 to 12.

The present invention further provides a process for forming the polymerizable compound according to the invention, which process comprises
(a) reacting at least one polymeric polyol having two or more repeating units and two or more hydroxyl groups with at least one diisocyanate, then
(b) reacting the product with at least one alcohol or amine comprising a fluorinated organic moiety of 1 to 18 carbon atoms and 35 to 85 wt % of fluorine, and then
(c) reacting the product with an organic compound comprising a polymerizable building-block group.

The reaction can be carried out in the absence of solvents to particular advantage. In this case, the polymeric polyol used can itself act as solvent, for example polycarbonate diols having a molecular weight of 1000 to 2000 g/mol, polyTHF having a molecular weight of 1000 to 2000 g/mol, especially 1700 g/mol, and polyethylene glycol having a molecular weight of 500 to 1500 g/mol, especially 750 g/mol. To obtain low molecular weight polymeric polyols, for example polyTHF having a molecular weight of 200 to 700 g/mol, it may be preferable to use Proglyde DMM (dipropylene glycol dimethyl ether) in addition. In this case, however, it is particularly advantageous that the amount, if needed at all, is distinctly smaller than according to the prior art.

The diisocyanate employed in step (a) is preferably a diisocyanate from the series 1,6-hexamethylene diisocyanate (HDI), 4,4′-, 2,4′- and/or 2,2′-diphenylmethane diisocyanate (MDI), 4,4′-dicyclohexylmethane diisocyanate (H12MDI), diphenylmethane diisocyanate (MDI), 2,4- and/or 2,6-tolylene diisocyanate (TDI) and 1-isocyanato-3,3,5-trimethyl-5-isocyanatomethylcyclohexane (IPDI). Further possibilities include m-xylene diisocyanate (MXDI), m- or p-tetramethylxylene diisocyanate (m-TMXDI, p-TMXDI), naphthalene 1,5-diisocyanate, cyclohexane 1,4-diisocyanate, hydrogenated xylylene diisocyanate (H6XDI), 1-methyl-2,4-diisocyanatocyclohexane, tetramethoxybutane 1,4-diisocyanate, butane 1,4-diisocyanate, 1,6-diisocyanato-2,2,4-trimethylhexane, 1,6-diisocyanato-2,4,4-trimethylhexane, 1-isocyanato-1-methyl-4(3)-isocyanatomethylcyclohexane (IMCI) and also 1,12-dodecane diisocyanate (C12DI), 4-dichlorophenyl diisocyanate, dicyclohexylmethane 4,4′-diisocyanate, m-phenylene diisocyanate, p-phenylene diisocyanate, 4-chloro-1,3-phenylene diisocyanate, 1,10-decamethylene diisocyanate, lysine alkyl ester diisocyanate, 3,3′-dimethyl-4,4′-diphenylmethane diisocyanate, xylylene diisocyanate, tetramethylxylylene diisocyanate, 1,5-tetrahydronaphthalene diisocyanate, methylenebis(cyclohexyl) 2,4′-diisocyanate and 4-methylcyclohexane 1,3-diisocyanate.

A particularly advantageous way to form the polymerizable compound of formula (I) is to use diisocyanates having two differently reactive isocyanate groups. This ensures that monofluorinated reaction products are formed selectively and that correspondingly, if at all, only an extremely small fraction of bifluorinated and/or nonfluorinated reaction products is obtained. This in turn has the consequence that, in the subsequent step of forming the polymerizable compound of formula (I), there is less by-product formation and almost complete incorporation of the fluorinated compounds in the polymer and correspondingly also in the network of the cured coating. It is therefore particularly preferable to use 1-isocyanato-3,3,5-trimethyl-5-isocyanatomethylcyclohexane.

Step (b) can employ especially the fluorinated alcohols and/or fluorinated amines described in respect of formula (I).

The polymeric polyol in step (c) is again preferably at least one from the group consisting of polyesters, polyalkylene ethers, polycarbonates, polyester carbonates, polyacetals, polyurethane polyols and polysiloxane polyol. Polyesters, polyalkylene ethers, polyester carbonates and polycarbonates are preferred polymeric polyols. The polymeric polyols described in respect of formula (I) are suitable.

The organic compound comprising a polymerizable building-block group is preferably at least one from the series acrylic acid, methacrylic acid, hydroxyalkyl acrylate, hydroxyalkyl methacrylate, dialkanolamine, hydroxyalkylcaprolactone acrylate, hydroxyalkylcaprolactone methacrylate, hydroxycycloalkyl methacrylate, hydroxycycloalkyl acrylate and hydroxyalkylmaleimide, preferably hydroxyalkyl methacrylate, dialkanolamine, hydroxyalkylcaprolactone methacrylate and tert-butylaminoethyl methacrylate.

Hydroxyethyl methacrylate and tert-butylaminoethyl methacrylate are especially preferred.

The present invention further provides an alternative process, which comprises
(a) reacting at least one alcohol or amine comprising a fluorinated organic moiety of 1 to 18 carbon atoms and 35 to 85 wt % of fluorine with at least one diisocyanate, and then
(b) reacting the product with at least one polymeric polyol comprising two or more repeating units and also one or more than one hydroxyl group and a polymerizable moiety.

The polymeric polyol comprising two or more repeating units and also one or more than one hydroxyl group and a polymerizable moiety may preferably be a polyglycol methacrylate.

Processes for forming polyglycol methacrylates are known from DE19602035 for example. Anhydrous polyethylene glycol methacrylates having molecular weights between 700 and 1000 g/mol are particularly suitable.

The present application further provides a polymer comprising the polymerizable compounds of the present invention. The polymer may more particularly be a comb-shaped polymer, in which case the polymerizable group in formula (I) becomes incorporated in the main chain of the polymer and the remainder, comprising the group R_{f}, acts as side chains.

The polymer comprising the polymerizable compounds of the present invention may be more particularly a copolymer with at least one further monomer. Various compounds are useful as further monomers, depending on the polymerizable group in formula (I).

When the polymerizable group is an unsaturated double bond, possibilities include for example at least one compound from the series ethyldiglycol acrylate, 4-tert-butylcyclohexyl acrylate, dihydrocyclopentadienyl acrylate, lauryl (meth)acrylate, phenoxyethyl (meth)acrylate, isobornyl (meth)acrylate, dimethylaminoethyl (meth)acrylate, cyanoacrylates, citraconate, itaconate and derivatives thereof, (meth)acrylic acid, methyl (meth)acrylate, ethyl (meth)acrylate, n-propyl (meth)acrylate, isopropyl (meth)acrylate, n-butyl (meth)acrylate, isobutyl (meth)acrylate, tert-butyl (meth)acrylate, n-pentyl (meth)acrylate, n-hexyl (meth)acrylate, cyclohexyl (meth)acrylate, n-heptyl (meth)acrylate, n-octyl (meth)acrylate, 2-propylheptyl (meth)acrylate, 2-ethylhexyl (meth)acrylate, nonyl (meth)acrylate, decyl (meth)acrylate, isodecyl (meth)acrylate, dodecyl (meth)acrylate, phenyl (meth)acrylate, tolyl (meth)acrylate, benzyl (meth)acrylate, 2-methoxyethyl (meth)acrylate, 3-methoxybutyl (meth)acrylate, 2-hydroxyethyl (meth)acrylate, 2-hydroxypropyl (meth)acrylate, stearyl (meth)acrylate, glycidyl (meth)acrylate, 2-aminoethyl (meth)acrylates, γ-(methacryloyloxypropyl)trimethoxysilane, ethylene oxide adducts of (meth)acrylic acid, trifluoromethylmethyl (meth)acrylate, 2-trifluoromethylethyl (meth)acrylate, 2-perfluoroethylethyl (meth)acrylate, 2-perfluoroethyl-2-perfluorobutylethyl (meth)acrylate, 2-perfluoroethyl (meth)acrylate, perfluoromethyl (meth)acrylate, diperfluoromethylmethyl (meth)acrylate, 2-perfluoromethyl-2-perfluoroethylmethyl (meth)acrylate, 2-perfluorohexylethyl (meth)acrylate, 2-perfluorodecylethyl (meth)acrylate and 2-perfluorohexadecylethyl (meth)acrylate. Particular preference is given to poly(ethylene glycol) methyl ether (meth)acrylate, (meth)acrylic acid and methyl methacrylate.

Further possibilities include, for example, isoprenol or hydroxybutyl vinyl ether when the polymerizable group is an unsaturated double bond, depending on the intended purpose. Further possibilities include mono- and polyunsaturated hydrocarbyl monomers, vinyl esters (e.g., vinyl esters of C₁-C₆ saturated monocarboxylic acids), vinyl ethers, monoethylenically unsaturated mono- and polycarboxylic acids and alkyl esters of these mono- and polycarboxylic acids (e.g., acrylic esters and methacrylic esters such as for instance C₁-C₁₂ alkyl and especially C₁-C₄ alkyl esters), amino monomers and nitriles, vinylidenes and alkylvinylidenes and amides of unsaturated carboxylic acids. Further possibilities include unsaturated hydrocarbyl monomers comprising styrene, compounds (e.g., styrene, carboxylated styrene and alpha-methylstyrene), ethylene, propylene, butylene and conjugated dienes (butadiene, isoprene and copolymers of butadiene and isoprene). Useful vinyl and halovinylidene monomers include vinyl chloride, vinylidene chloride, vinyl fluoride and vinylidene fluoride. Examples of vinyl esters include aliphatic vinyl esters, for instance vinyl formate, vinyl acetate, vinyl propionate, vinyl butyrate, vinyl isobutyrate, vinyl valerate, vinyl caproate and allyl esters of saturated monocarboxylic acids such as allyl acetate, allyl propionate and allyl lactate. Suitable vinyl ethers include methyl vinyl ether, ethyl vinyl ether and n-butyl vinyl ether. Typical vinyl ketones include methyl vinyl ketones, ethyl vinyl ketones and isobutyl vinyl ketones. Examples of dialkyl esters of monoethylenically unsaturated dicarboxylic acids are dimethyl maleate, diethyl maleate, dibutyl maleate, dioctyl maleate, diisooctyl maleate, dinonyl maleate, diisodecyl maleate, ditridecyl maleate, dimethyl fumarate, diethyl fumarate, dipropyl fumarate, dibutyl fumarate, dioctyl fumarate, diisooctyl fumarate, didecyl fumarate, dimethyl itaconate, diethyl itaconate, dibutyl itaconate and dioctyl itaconate. The monoethylenically unsaturated monocarboxylic acids are in particular acrylic acid, methacrylic acid, ethacrylic acid and crotonic acid. Maleic acid, fumaric acid, itaconic acid and citric acid may be mentioned as monoethylenically unsaturated dicarboxylic acids. Useful monoethylenically unsaturated tricarboxylic acids for the purposes of the present invention include for example aconitic acid and its halogen-substituted derivatives. It is further possible to use the anhydrides and esters of the aforementioned acids (e.g., maleic anhydride and citric anhydride). Useful nitriles of ethylenically unsaturated mono-, di- and tricarboxylic acids include for example acrylonitrile, α-chloroacrylonitrile and methacrylonitrile. The amides of carboxylic acids may be acrylamides, methacrylamides and other α-substituted acrylamides and N-substituted amides, e.g. N-methylolacrylamide, N-methylolmethylacrylamide, alkylated N-methylolacrylamides and N-methylolmethacrylamides (e.g., N-methoxymethylacrylamide and N-methoxymethylmethacrylamide). Useful amino monomers include substituted and unsubstituted aminoalkyl acrylates, hydrochloride salts of amino monomers and methacrylates such as for instance β-aminoethyl acrylate, β-aminoethyl methacrylate, dimethylaminomethyl acrylate, β-methylaminoethyl acrylate and dimethylaminomethyl methacrylate. Useful cationic monomers for the purposes of the present invention include α- and β-ethylenically unsaturated compounds which are suitable for chain growth addition polymerization and comprise primary, secondary or tertiary amino groups, e.g., dimethylaminoethyl methacrylate, dimethylaminoneopentyl acrylate, dimethylaminopropyl methacrylate and tert-butylaminoethyl methacrylate or organic or inorganic salts of these compounds and/or alkylammonium compounds such as for instance trimethylammonioethyl methacrylate chloride, diallyldimethylammonium chloride, β-acetamidodiethylaminoethyl acrylate chloride and methacrylamidopropyltrimethylammonium chloride. These cationic monomers can be used alone or combined with the aforementioned further monomers. β-Hydroxyethyl (meth)acrylates, β-hydroxypropyl (meth)acrylates and γ-hydroxypropyl (meth)acrylates may additionally be mentioned as examples of hydroxyl-containing monomers.

The compounds used in the above-described process can be employed as diisocyanate and also alcohol or amine comprising a fluorinated organic moiety of 1 to 18 carbon atoms and 35 to 85 wt % of fluorine.

When the polymerizable group in formula (I) comprises hydroxyl and/or amino groups, these are preferably reacted with an isocyanate component to form polymers. The isocyanate component is preferably an aliphatic, cycloaliphatic, araliphatic and/or aromatic compound, preferably a diisocyanate or triisocyanate, including mixtures of these compounds. It is preferable here for 1,6-hexamethylene diisocyanate (HDI), HDI uretdione, HDI isocyanurate, HDI biuret, HDI allophanate, 1-isocyanato-3,3,5-trimethyl-5-isocyanatomethylcyclohexane (IPDI), 2,4- and/or 2,6-tolylene diisocyanate (TDI) and/or 4,4′-, 2,4′- and/or 2,2′-diphenylmethane diisocyanate (MDI), polymeric MDI, carbodiimide-modified 4,4′-MDI, m-xylylene diisocyanate (MXDI), m- or p-tetramethylxylylene diisocyanate (m-TMXDI, p-TMXDI), 4,4′-dicyclohexylmethane diisocyanate (H12MDI), naphthalene 1,5-diisocyanate, cyclohexane 1,4-diisocyanate, hydrogenated xylylene diisocyanate (H6XDI), 1-methyl-2,4-diisocyanatocyclohexane, tetramethoxybutane 1,4-diisocyanate, butane 1,4-diisocyanate, 1,6-diisocyanato-2,2,4-trimethylhexane, 1,6-diisocyanato-2,4,4-trimethylhexane, 1-isocyanato-1-methyl-4(3)-isocyanatomethylcyclohexane (IMCI) and also 1,12-dodecane diisocyanate (C12DI) to be concerned. Also 4-dichlorophenyl diisocyanate, dicyclohexylmethane 4,4′-diisocyanate, m-phenylene diisocyanate, p-phenylene diisocyanate, 4-chloro-1,3-phenylene diisocyanate, 1,10-decamethylene diisocyanate, lysine alkyl ester diisocyanate, 3,3′-dimethyl-4,4′-diphenylmethane diisocyanate, xylylene diisocyanate, tetramethylxylylene diisocyanate, 1,5-tetrahydronaphthalene diisocyanate, triisocyanatotoluene, methylenebis(cyclohexyl) 2,4′-diisocyanate and 4-methylcyclohexane 1,3-diisocyanate can be concerned. Polyisocyanates having two or three isocyanate groups per molecule are also suitable. But mixtures of polyisocyanates can also be concerned, in which case the average NCO functionality of the isocyanate component in the mixture can be especially in the range from 2.0 to 2.3, 2.2 to 2.4, 2.6 to 2.8 or 2.8 to 3.0. Derivatized polyisocyanates can likewise be used, examples being sulfonated isocyanates, blocked isocyanates, isocyanurates and biuret isocyanates.

The polymer of the present invention is preferably a polyacrylate, polymethacrylate, polyurethane or polyurea.

In one particularly preferred embodiment, the polymer of the present invention is used for coating a surface, more preferably for permanent water- and dirt-repellent surface coating. Such applications preferably comprise coatings with a so called “anti-icing” effect under specific climatic and weather conditions (temperatures between −10 and +5° C., wind, air humidity), particularly for temporary or permanent fields of use such as but not limited to aircraft wing construction, on a roof or doorway or for rotor and wind mill blades.

It was found altogether that, surprisingly, the polymerizable compounds of formula (I) lead even at low use levels and a lower fluorine content to outstanding water- and dirt-repellent properties in the coatings produced therefrom. The products formed from the polymerizable compounds of formula (I) possess very good mechanical and photochemical stability. The coatings produced further have a good appearance and a high hardness and also good chemical resistance. More particularly, the fluorinated monomers of the present invention are obtainable in a simple and economical manner.

The examples which follow illustrate the advantages of the present invention.

### EXAMPLES

### Example 1

### Preparation of Inventive Fluoromonomer 1 (F-polycarbonate1000 diol)

53.35 g of isophorone diisocyanate (IPDI) are initially charged to a 500 ml three-neck flask equipped with stirrer, reflux condenser and thermometer together with 0.04 g of dibutyltin dilaurate and heated to 40° C. 120.00 g of polycarbonate (MW=1000 g/mol) are added, and the reaction mixture is heated to 70° C. and stirred at 70° C. for 1 hour. 2-(Perfluorohexyl)ethanol (DAIKIN A-1620) is added in two portions 20 minutes apart (total amount: 43.69 g). Following each addition of the fluoroalcohol, the temperature of the reaction mixture rises by about 10° C. The temperature of the reaction mixture is adjusted to 50° C. and the NCO content is checked following a reaction time of 30 minutes. Once the theoretical NCO content of 2.32 wt % is reached, the reaction mixture is slowly added to diethanolamine (10.72 g).

### Example 2

### Preparation of Inventive Fluoromonomer 2 (F-polyTHF1000 Methacrylate)

85.45 g of isophorone diisocyanate (IPDI) are initially charged to a 500 ml three-neck flask equipped with stirrer, reflux condenser and thermometer together with 0.04 g of dibutyltin dilaurate and heated to 40° C. 192.25 g of polyTHF (MW=1000 g/mol) are added, and the reaction mixture is heated to 60° C. and stirred at 70° C. for 1 hour. 2-(Perfluorohexyl)ethanol (DAIKIN A-1620) is added in three portions 20 minutes apart (total amount: 70.00 g). Following each addition of the fluoroalcohol, the temperature of the reaction mixture rises by about 10° C. The temperature of the reaction mixture is adjusted to 50° C. and the NCO content is checked following a reaction time of 30 minutes. Once the theoretical isocyanate value of 2.32 wt % is reached, 26.24 g of 2-hydroxyethyl methacrylate (HEMA) are added and the reaction is continued at 55° C. to the end-point (0 wt % NCO) (HEMA in excess, 95% of OH groups are reacted). The product is diluted with dipropylene glycol dimethyl ether (41.55 g) so that the solids content is 90 wt %.

### Example 3

### Preparation of Inventive Fluoromonomer 3 (F-Polyglycol Methacrylate)

42.74 g of isophorone diisocyanate (IPDI) are initially charged to a 500 ml three-neck flask equipped with stirrer, reflux condenser and thermometer together with 0.1 g of dibutyltin dilaurate and heated to 40° C. 2-(Perfluorohexyl)ethanol (DAIKIN A-1620) is added in three portions 20 minutes apart (total amount: 70.00 g). Following each addition of the fluoroalcohol, the temperature of the reaction mixture rises by about 10° C. The temperature of the reaction mixture is adjusted to 50° C. and the NCO content is checked following a reaction time of 30 minutes. Once the theoretical isocyanate value of 7.16 wt % is reached, 147.08 g of MPEG MA 750 (poly(ethylene glycol) methyl ether methacrylate) are added and the reaction is continued at 60° C. to the end-point (0 wt % NCO) (MPEG MA 750 in excess, 98% of OH groups are reacted).

### Example 4

### Preparation of Fluoromonomer 4 (F-IPDI-HEMA) (Comparative Example)

91.58 g of isophorone diisocyanate (IPDI) are initially charged to a 500 ml three-neck flask equipped with stirrer, reflux condenser and thermometer together with 0.04 g of dibutyltin dilaurate and heated to 40° C. 2-(Perfluorohexyl)ethanol (DAIKIN A-1620) is added in three portions 20 minutes apart (total amount: 150.0 g). Following each addition of the fluoroalcohol, the temperature of the reaction mixture rises by about 10° C. The temperature of the reaction mixture is adjusted to 45° C. and the NCO content is checked following a reaction time of 30 minutes. Once the theoretical isocyanate value of 7.16 wt % is reached, 2-hydroxyethyl methacrylate (HEMA) is added and the reaction is continued at 35° C. to the end-point (0 wt % NCO) (HEMA in excess, only 80-90% of OH groups are reacted).

### Example 5

5.51 g of poly(ethylene glycol) methyl ether methacrylates (MPEG 475 MA), 38.20 g of dipropylene glycol dimethyl ether, 4.55 g of methacrylic acid, 69.09 g of methyl methacrylate and 18.02 g of fluoromonomer 2 are initially charged to a 250 ml three-neck flask equipped with stirrer, reflux condenser, thermometer under nitrogen and heated to 75° C. Reactor temperature is adjusted to 75° C. 5.33 g of 2-mercaptoethanol are added. 0.42 g of initiator (VAZO-67) is dissolved in 16.61 g of toluene and the 3% initiator solution is added linearly using a perfusor pump at a flow rate of 5.44 ml/h over a period of 3 h. On completion of the metered addition, the reaction mixture is stirred at 70° C. for 1 hour. The reaction mixture is cooled down and the pH is adjusted to 6.5-7 with NaOH (50 wt %). The slightly cloudy, white solution thus obtained has a solids content of 63.2 wt % and 2.5 wt % fluorine content in the solid material.

### Example 6

5.51 g of poly(ethylene glycol) methyl ether methacrylates (MPEG 475 MA), 40.00 g of dipropylene glycol dimethyl ether, 4.55 g of methacrylic acid, 69.09 g of methyl methacrylate and 6.25 g of fluoromonomer 4 are initially charged to a 250 ml three-neck flask equipped with stirrer, reflux condenser, thermometer under nitrogen and heated to 75° C. Reactor temperature is adjusted to 75° C. 5.33 g of 2-mercaptoethanol are added. 0.42 g of initiator (VAZO-67) is dissolved in 16.61 g of toluene and the 3% initiator solution is added linearly using a perfusor pump at a flow rate of 5.44 ml/h over a period of 3 h. On completion of the metered addition, the reaction mixture is stirred at 70° C. for 1 hour. The reaction mixture is cooled down and the pH is adjusted to 6.5-7 with NaOH (50 wt %). The slightly cloudy, white solution thus obtained has a solids content of 58.3 wt % and 2.5 wt % fluorine content in the solid material.

The polyacrylates (polyacrylate products from Examples 5 and 6) were blade coated onto a presentation foil using a 250 μm bar. The solvent-containing films were stored in vacuo (2-10 mbar) for 3 hours at RT before being measured for dynamic contact angle as well as advancing and receding angle of machine oil.

### Contact Angle Measurement:

Dynamic contact angle measurements: Dynamic contact angles are determined using a standardized setup provided by Krüss (“Drop Shape Analysis Instrument”, DSA 10). The instrument monitors the shadow of a liquid droplet placed on a solid substrate by means of a video camera. In this way, the geometry of the droplet and time-dependent changes in its shape are directly accessible. Basically, any combination of liquid and substrate can be investigated by this method. The drop shape and the contact angle with the substrate are evaluated via automated and computerized image analysis (corresponding software “DSA”).

In general, contact angles can be measured either under static (droplet with a given constant volume) or dynamic conditions. In the latter case, the interface between the droplet and the substrate is tracked while the droplet volume is gradually increased (advancing contact angle) or decreased (receding contact angle), which in essence corresponds to wetting and dewetting, respectively, of the substrate. Surfaces that are stain-resistant and repel liquids usually exhibit high receding angles. The receding angle is a direct measure for how readily a wetted surface can be dewetted again. For idealized, chemically homogeneous and smooth surfaces, no distinct hysteresis is expected between advancing and receding angles. Significant differences in this parameter indicate heterogeneities in the morphology or chemistry of the surface.

Advancing contact angle. For measuring advancing contact angles, a small drop (3-5 μL) of oil (e.g. machine oil (preferred), sunflower oil or paraffin oil) is placed on the sample films with the aid of a thin syringe needle (typical inner diameter: 0.5 mm), after all equipment has been equilibrated at 23° C. The syringe needle is left submerged in the oil drop. In the following, the volume of the droplet is gradually increased by adding further oil through the needle at a rate of 20 μL/min. This is done either manually with a conventional disposable syringe, or automatically via an attached electrical syringe pump. At the beginning of the measurement, the optical system is adjusted to focus on the contour of the droplet. Then, as the droplet is enlarged, its shadow is continuously monitored. In this way, the extension of the droplet is traced over a period of 10-20 s, until a final volume of 10-15 μL is reached.

The shape of the oil drop in individual video frames is evaluated already during the measurements by means of on-line digital image analysis. To that end, the contour of the drop is approximated by polynomial fitting. This procedure has proven to be a reliable method to obtain correct results over a wide range of contact angles. Finally, the contact angle is determined by applying tangents to the fitted contour line. Resulting values are saved as a function of time and, thus, also of droplet volume. Typically, contact angles are given as an average (with corresponding standard deviation) of values measured at different times, since in principle the contact angle should not depend on the volume of the droplet. This, however, does not necessarily apply for the initial stages of volume increase, since the immersed needle may still have an influence on the droplet shape at the contact point. Therefore, data collected in this regime are not considered in the evaluations.

Receding contact angle. In order to determine receding contact angles, a relatively large drop (diameter of about 6 mm) of oil is formed on the substrate, while again the tip of the used needle remains within the liquid phase. Subsequently, oil is continuously removed from the drop using a syringe. Common volume decrease rates are in the range of 20 μL/min. As in the case of the advancing angle, the gradual reduction of the droplet volume is recorded by a video camera. As soon as distinct regression of the liquid across the substrate surface is distinguished, the drop shape and, with it, the contact angle are determined by polynomial fitting over a sampling period of 10-20 s, while finally all measured values are averaged. Data treatment and evaluation is performed as described for the advancing contact angle.

TABLE 1
Composition of perfluoroalkyl-containing polycarboxylate
ethers of Examples 5 and 6 and parameters

| | |
|---|---|
| Example 5 | Example 6 |
| (fluoro- | (comparative example; |
| monomer 2) | fluoromonomer 4) |

| | | | | |
|---|---|---|---|---|
| MPEG methacrylate | 5.7 | wt % | 6.5 | wt % |
| methyl methacrylate | 71.1 | wt % | 80.9 | wt % |
| methacrylic acid | 4.7 | wt % | 5.3 | wt % |
| fluoromonomer | 18.5 | wt % | 7.3 | wt % |
| fluorine content | 2.5 | wt % | 2.5 | wt % |
| FK theory | 72.9 | wt % | 69.5 | wt % |
| FK NP | 63.2 | wt % | 58.3 | wt % |

| | | |
|---|---|---|
| viscosity [mPas] | 45860 | 20450 |
| Mw [g/mol] | 3322 | 1969 |
| Mn [g/mol] | 1501 | 1259 |
| appearance | cloudy | cloudy |
| contact angle H₂O [°] | 98.1 | 94.0 |
| contact angle CH₂I₂ [°] | 32.8 | 89.2 |

Key to Table 1:
M_{w} = weight-average molecular weight measured by gel permeation chromatography (GPC) versus a PEG standard
Mₙ = number-average molecular weight measured by gel permeation chromatography (GPC) versus a PEG standard
FK NP = solids content on completion of polymerization reaction
Viscosity was measured at 21° C. using a CAP 2000 HAAKE rotary viscometer, spindle 4, RPM 12.
Contact angle H₂O: measured contact angle versus water
Contact angle CH₂I₂: measured contact angle versus diiodomethane
Comparative example: fluoromonomer 4 based on 2-(perfluorohexylethanol)/IPDI/HEMA

### Advancing and Receding Angles Versus Machine Oil:

Surfaces which are to be dirt repellent and to bead off liquids must form a large receding angle. It indicates the dewettability of a once wetted surface. Smooth surfaces with ideal chemical homogeneity would not be expected to form a large hysteresis between the advancing angle and the receding angle. A difference indicates heterogeneities in the morphology or surface chemistry.

TABLE 2
Result of machine oil advancing and receding angle measurements

| Coating | Advancing angle | Receding angle | Hysteresis |
|---|---|---|---|
| Coating of Example 5 | 99.5° | 18.9° | 80.6 |
| Coating of Example 6 | 84.9° | 15.8° | 69.1 |
| (comparative example) | | | |

Example 5 shows distinctly elevated advancing and receding angles compared with Example 6 for the same fluorine content, suggesting improved dirt repellency.

### Time of Flight Secondary Ion Mass Spectrometry:

Time of flight secondary ion mass spectrometry (TOF-SIMS) is a powerful surface analysis technique that allows chemical characterization of the first atomic layers of any vacuum stable solid surface. Being a fragmenting mass spectrometry technique, it provides a wealth of chemical information and allows identifying compounds with very high sensitivity. Furthermore, due to its surface sensitivity, the packing density and orientation of functional groups on a surface can be judged. SIMS is not quantitative without prior calibration on each sample system due to a modulation of ionization probabilities by the composition of the surrounding matrix. However, if the matrix is kept sufficiently constant, SIMS intensities for certain mass fragments and especially ratios of integrals of characteristic mass peaks can be used for comparison.

Static TOF-SIMS spectra were recorded using a TOF-SIMS V spectrometer (IonTOF GmbH, Germany). A pulsed mass-filtered primary ion beam of 25 keV singly charged bismuth (Bit) was used. This primary ion beam, resulting in a spot size of typically 5 μm on the sample surface, was raster scanned over an area of 500×500 μm to record spectra of positive and negative secondary ions. The primary ion dose density was always kept well below 10⁻¹² ions/cm² and thus in the static SIMS regime. To prevent charging of the sample surface, a low-electron energy flood gun was used.

The samples (polyacrylate products from Examples 5 and 6) were prepared as films by knife-coating with 250 μm wet layer thickness on PET films. Samples were vacuum dried (8-10 mbar) at RT for 3 hours before measurements.

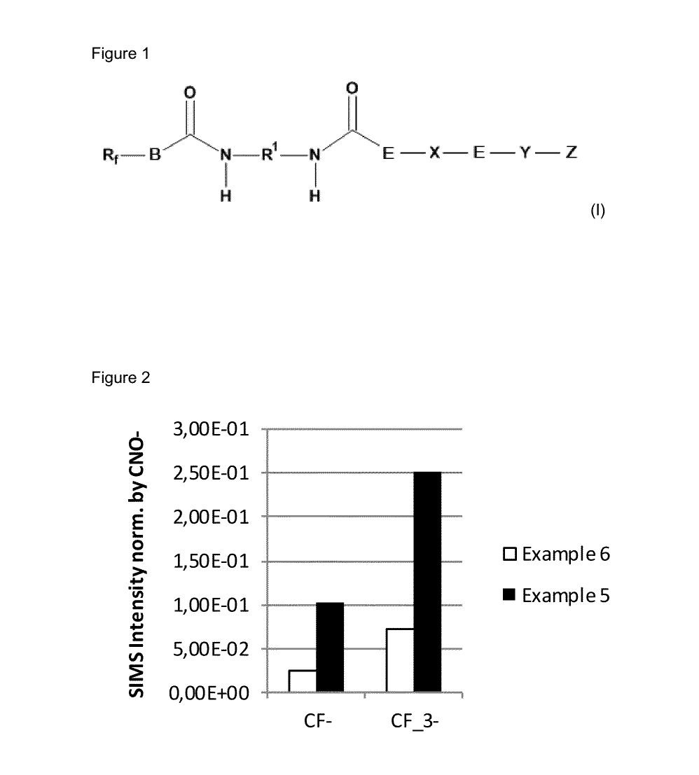

As a result shown in FIG. 2 and table 3, the sample comprising the fluoro monomer 2 (Example 5) shows an increased accumulation of fluorine on the surface compared to the reference sample (Example 6). While not to be bound by theory, it is believed that the higher accumulation of fluorine in case of Example 5 is due to a higher mobility of fluoro-modified side chains compared to Example 6 (reference).

TABLE 3
Integrated Secondary Ion Intensity

| Integrated Secondary Ion In- | Example 6 | Example 5 |
|---|---|---|
| tensity Normalized by CNO− | (reference sample) | (fluoro monomer 2) |
| CF− | 2.42E−02 | 1.03E−01 |
| CF₃− | 7.22E−02 | 2.52E−01 |

### Example

### Easy-to-Clean Coating

In the following section the coating properties of a formulation (Example 11) comprising a fluoro-modified polyurethane dispersion (Example 7) prepared via covalent incorporation of a fluoro-modified diol-building block (Example 8) are described. Example 11 is an aqueous, pigmented two component formulation based on a hydroxyl-functional acrylic dispersion, which is cured with a hydrophilic, aliphatic isocyanate. The product is applied as a top coat in the industrial sector for medium to heavy loads.

### Preparation of Example 7

### Fluoro-Modified Polyurethane Dispersion

A four-necked flask equipped with a KPG-stirrer, reflux condenser, thermometer and nitrogen coverage is charged with a mixture of 136.77 g of fluoro-modified diol component (cf. Example 8), 163.14 g isophorone diisocyanate (IPDI) in presence of 0.1 g of dibutyltindilaureate (DBTL) as a catalyst and 105.00 g of a polyesterdiol with a hydroxy-value of ca. 56.1 mg KOH/g under nitrogen atmosphere and stirred for 1.5 h at 80-90° C. After the addition of 14.95 g 1,4-butanediol and 19.10 g dimethylol propionic acid (DMPA®), the mixture is dissolved in 28.64 g of di(propylene glycol) dimethyl ether and stirred for 1 h at 80-90° C. under nitrogen atmosphere until the calculated NCO-content of 5.46 wt % is reached. The conversion is monitored via acidimetric titration. The pre-polymer (484.86 g) is mixed under intensive stirring in a mixture of 644.51 g water and 12.97 g triethylamine. In addition, 28.93 g ethylenediamine (as a 16.7 wt % aqueous solution) was added to the dispersion in order to accomplish chain elongation. As a result, a stable polyurethane dispersion with a solid content of 38 wt %, a charge density of 30.50 meq/100 g and a fluorine content of 1.0 wt % is obtained.

### Preparation of Example 8

### Fluoro-Modified Diol

A four-necked flask equipped with a KPG-stirrer, reflux condenser, thermometer and nitrogen coverage is charged with a mixture of 88.92 g isophorone diisocyanate (IPDI), 400.0 g of a polyesterdiol with a hydroxy-value of ca. 56.1 mg KOH/g in presence of 0.04 g of dibutyltindilaureate (DBTL) as a catalyst. The mixture is dissolved in 145.71 g of di(propylene glycol) dimethyl ether under nitrogen atmosphere and stirred for 1.0 h at 75° C. A fluoro-alcohol (2-perfluorohexyl ethanol, 72.84 g) is added and the mixture is stirred for 30 min at 75° C. until a NCO-content of 1.19 wt % is reached. The mixture is cooled down to 40° C. and added slowly to 20.06 g of diethanolamine. Conversion is completed when the NCO-content has reached the zero-value.

### Easy-to-Clean Application Tests

### Preparation of Example 9

### Basic Formulation

To a hydroxyl functionalized acrylate dispersion (solid content of 37-39 wt %, OH-content as supplied of 1.8 wt %, minimum film-forming temperature of 15° C., pH value 7.5-8.5), polyurethane thickeners with Newtonian flow behavior, a defoamer (based on silica derivatives, mineral oil and esters) and a matting agent, based on polycondensate plastics with a particle size d50 of approx. 6.3 μm are added under stirring for 15 min at 16 m/s. In addition, preserving agents, an additive for substrate wetting (based on polyether-modified siloxane) and water are added under stirring at 5 m/s and the mixture is finally stirred for 10 min at 5 m/s.

TABLE 4
Composition of Example 9

| Pos. | Raw material | Quantity [%] |
|---|---|---|
| 1 | Hydroxyl functional acrylate dispersion | 75.00 |
| | with following properties: | |
| | Solid-content 37-39 wt % | |
| | OH-content as supplied 1.8 wt % | |
| | MFFT (minimum film-forming temperature) | |
| | 15° C. pH value 7.5-8.5 (Alberdingk AC | |
| | 27401; Alberdingk & Boley GmbH) | |
| 2 | Polyurethane thickeners with Newtonian flow | 1.90 |
| | behavior (Rheovis PU 1214; BASF SE) | |
| 3 | Defoamer, based on silica derivatives, mineral oil | 1.00 |
| | and esters (Drewplus T-4201; Ashland Inc.) | |
| | Matting agent, based on Polycondensate plastics | 2.00 |
| | with a particle size d50 approx. 6.3 μm | |
| | (Deuteron MK, Deuteron GmbH) | |
| 4 | Preserving agents (Parmetol A 28 S, Schülke & | 0.10 |
| | Mayr GmbH) | |
| 5 | Additive for substrate wetting, based on | 0.30 |
| | Polyether-modified siloxane (BYK-345, BYK | |
| | Chemie GmbH) | |
| 6 | Water | 19.70 |
| Σ | | 100.00 |

Example 10 (reference formulation) is prepared starting from Example 9 (basic formulation) via the addition of a color paste mix (RAL 7032, pebble grey) under stirring at 5 m/s and additional stirring for 10 min at 5 m/s. After that, an aliphatic isocyanate (NCO content of 21.6 wt %, dynamic viscosity of 1100 mPa*s) is added under stirring (5 m/s stirring speed) and finally stirred for 5 min at 5 m/s.

TABLE 5
Composition of Example 10 (reference)

| Pos. | Raw material | Quantity [%] |
|---|---|---|
| 1 | Example 9 | 64.00 |
| 2 | Color pastes mix in RAL 7032 (pebble grey) | 16.00 |
| | (Chromachem WAB, Chromaflo Technologies | |
| | B.V.) | |
| 3 | Aliphatic isocyanate with following properties: | 20.00 |
| | NCO content 21.6 wt % | |
| | Dynamic viscosity 1100 mPa * s | |
| | Solid-content 100% | |
| | (Easaqua X M 501, Perstorp Holding AB) | |
| Σ | | 100.00 |

Example 1 (fluoro-modified formulation) is prepared starting from Example 9 (basic formulation) via the addition of a color paste mix (RAL 7032, pebble grey) and a fluoro modified dispersion (Example 7) under stirring at 5 m/s and additional stirring for 10 min at 5 m/s. After that, an aliphatic isocyanate (NCO content of 21.6 wt %, dynamic viscosity of 1100 mPa*s) is added under stirring (5 m/s stirring speed) and finally stirred for 5 min at 5 m/s. The final fluorine content is 0.06 wt %.

TABLE 6
Composition of Example 11 (fluoro-modified)

| Pos. | Raw material | Quantity [%] |
|---|---|---|
| 1 | Example 9 | 57.60 |
| 2 | Color pastes mix in RAL 7032 (pebble grey) | 16.00 |
| | (Chromachem WAB, Chromaflo Technologies | |
| | B.V.) | |
| 3 | Example 7 (fluoro modified dispersion) | 6.40 |
| 4 | Aliphatic isocyanate with following properties: | 20.00 |
| | NCO content 21.6 wt % | |
| | Dynamic viscosity 1100 mPa * s | |
| | Solid-content 100% | |
| | (Easaqua X M 501, Perstorp Holding AB) | |
| Σ | | 100.00 |

### Specimen Preparation

The formulations from Examples 10 and 11 are applied on a substrate (MASTERTOP BC 375 N; BASF Construction Chemicals Europe AG (Solvent free, pigmented 2K PU leveling coating, applied with a consumption of 2.5 kg/m². Surface properties: non-absorbent, smooth and glossy)) consisting of a polyurethane based on a branched, castor oil based polyol, cross-linked with a 4,4-MDI with a Shore-D-Hardness after 28 days of 70 and elongation at break of 10%. The consumption of formulation was 120 g/m² (applied by roller) each. Testing was performed at 23° C. and 50% relative humidity.

### Results Easy-to-Clean Application Tests

### Dirt Pickup

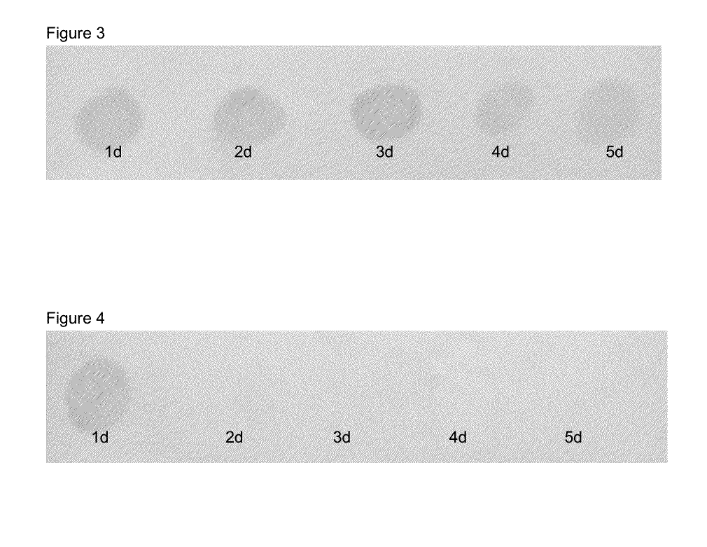

The dirt pickup is tested with an aqueous carbon black paste (5% in water). During the first 5 days after the application, 5 ml of the aqueous carbon black paste are placed for 8 hours on the top coat. Subsequently, the stain is removed with water and a paper towel (FIG. 3 and FIG. 4). The residue gives information about the dirt pickup especially in the first days after application.

FIG. 4 (Example 11 (fluoro-modified) on substrate (MASTERTOP BC 375 N)) shows that a fluoro-modification of only 0.06 wt % reduces dirt-pick-up significantly compared to FIG. 3 (Example 10 (reference) on substrate (MASTERTOP BC 375 N)) after the first day of application.

Wear resistance tests were performed according to DIN 52347 (Taber-Abraser). The wear is determined after 28 days of storage at room temperature under the following conditions: Wheel CS 10/500 g/1000 T. As a result, abrasion of the fluoro-modified coating on the substrate (MASTERTOP BC 375 N) is lower which indicates an improved performance in terms of abrasion properties.

TABLE 7
Abrasion properties

| Specimen | Abrasion after 28 days [mg] |
|---|---|
| Example 10 (reference) on substrate | 24 |
| (MASTERTOP BC 375 N) | |
| Example 11 on substrate | 19 |
| (MASTERTOP BC 375 N) | |

Sliding properties were tested according to DIN EN 13893. The coefficient of sliding friction is measured after 14 and 28 days No difference between the two formulations regarding the sliding properties can be observed, which again is an indication that the overall performance of the coating is maintained and fluoro-modification has no negative impact on sliding properties.

TABLE 8
Sliding friction properties

| | Coefficient of sliding friction | |
|---|---|---|
| Specimen | After 14 days | After 28 days |
| Example 10 (reference) on substrate | 0.45 | 0.46 |
| (MASTERTOP BC 375 N) | | |
| Example 11 on substrate | 0.46 | 0.44 |
| (MASTERTOP BC 375 N) | | |

The adhesion to the substrate according to DIN ISO 2409 is tested after 7 and 14 days by the Cross-Cut-Test. Both formulations show a very good adhesion to the substrate (MASTERTOP BC 375 N) which is one of the most important parameters for the final application as an industrial coating.

TABLE 9
Adhesion properties

| | Adhesion [GT] | |
|---|---|---|
| Specimen | After 7 days | After 14 days |
| Example 10 (reference) on substrate | 0 | 0 |
| (MASTERTOP BC 375 N) | | |
| Example 11 on substrate | 0 | 0 |
| (MASTERTOP BC 375 N) | | |

1.-15. (canceled)

16. A polymerizable compound of formula (I), 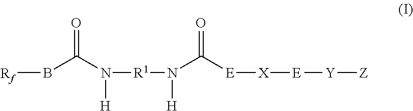
wherein
R_{f} is a monovalent fluorinated organic radical of 1 to 18 carbon atoms and 35 to 85 wt % of fluorine;
R¹ is a moiety from the group consisting of cyclic or acyclic alkylene of 5 to 14 carbon atoms, arylene, diarylenealkane and dialkylene-substituted aryl;
B is —O— or —N(R²)—;
R² is hydrogen, an aliphatic hydrocarbyl moiety of 1 to 12 carbon atoms or a monovalent fluorinated organic radical of 1 to 18 carbon atoms and 35 to 85 wt % of fluorine;
X is a polymeric polyol having two or more repeating units and after removal of two OH groups;
E is —O— or —N(R³)—;
R³ is a hydrocarbyl moiety of 1 to 12 carbon atoms or a hydrogen atom;
Y is a chemical bond or a divalent moiety of 2 to 20 carbon atoms; and
Z is an organic moiety comprising a polymerizable building-block group.

17. The polymerizable compound according to claim 16, wherein X is a building-block group of formula (II), 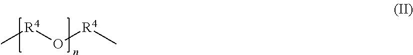
wherein each R⁴ may be the same or different and n represents an integer from 1 to 70.

18. The polymerizable compound according to claim 16, wherein X is a building-block group of formula (IV), 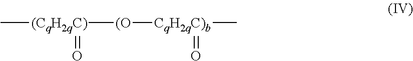
wherein each q represents independently for every (C_{q}H_{2q}(C═O)) unit an integer from 2 to 18 and is the same or different and b represents an integer between 1 and 30.

19. The polymerizable compound according to claim 16, wherein R¹ is selected from the group consisting of methylene, ethylene, propylene, butylene, pentamethylene, hexamethylene, octamethylene, 2-ethylhexamethylene, phenylene, tolylene, methylenebisphenyl, propylenebisphenyl, cyclohexylene, methylene- or propylenebiscyclohexyl moieties and a group obtained by removing the isocyanate groups from 1-isocyanato-3,3,5-trimethyl-5-isocyanatomethyl-cyclohexane.

20. The polymerizable compound according to claim 16, wherein Y is selected from the group consisting of methylene, ethylene, propylene, butylene, pentamethylene, hexamethylene, octamethylene, 2-ethylhexamethylene, phenylene, tolylene, methylenebisphenyl, propylenebisphenyl, cyclohexylene, methylenebiscyclohexyl, propylenebiscyclohexyl and a group obtained by removing the isocyanate groups from 1-isocyanato-3,3,5-trimethyl-5-isocyanatomethyl-cyclohexane.

21. The polymerizable compound according to claim 16 wherein R_{f} is at least one moiety of formula (V),
CF₃—(CF₂)_{z}—(CH₂)_{y}— (V)
wherein
each z is an integer from 0 to 16 and is the same or different, and
each y is an integer from 1 to 6 and is the same or different.

22. The polymerizable compound according to claim 16 wherein Z is a moiety from the group consisting of acrylate, methacrylate, oxyalkylacrylate, oxyalkylmethacrylate, dialkanolaminyl, oxyalkylcaprolactoneacrylate, oxyalkylcaprolactonemethacrylate, oxycycloalkylmethacrylate, oxycycloalkylacrylate and oxyalkylmaleimide.

23. The polymerizable compound according to claim 16 comprising a compound from the group consisting of formula (VI), formula (VII), formula (VIII) and formula (IX), 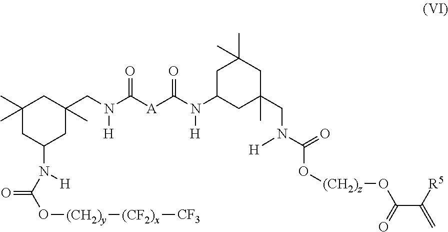
wherein
A is a building-block group of the formula —O—(CₘH₂ₘO)ₐ— where each m represents independently for every (CₘH₂ₘO) unit an integer from 2 to 4 and is the same or different and a represents an integer from 2 to 30,
R⁵ is hydrogen or methyl;
z is an integer from 1 to 5;
x is an integer from 0 to 9;
y is an integer from 2 to 12; 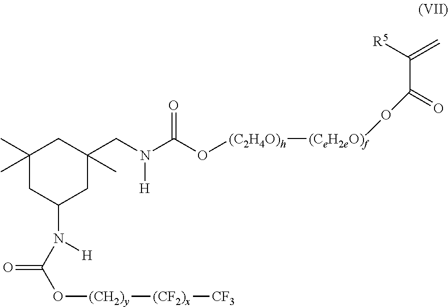
wherein
R⁵ is hydrogen or methyl;
e in each occurrence is 2 or 3 independently for every (CₑH₂ₑO) unit and is the same or different;
f is an integer from 0 to 150,
h is an integer from 0 to 50 subject to the proviso that the sum total of f and h is not less than 3;
x is an integer from 0 to 9;
y is an integer from 2 to 12; 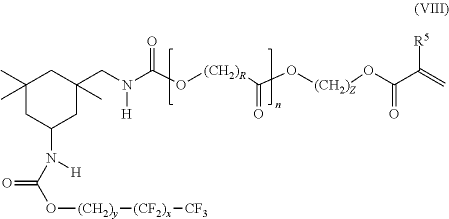
wherein
Z is an integer from 1 to 50;
R is an integer from 2 to 18;
n is an integer from 1 to 10;
x is an integer from 0 to 9;
y is an integer from 2 to 12; 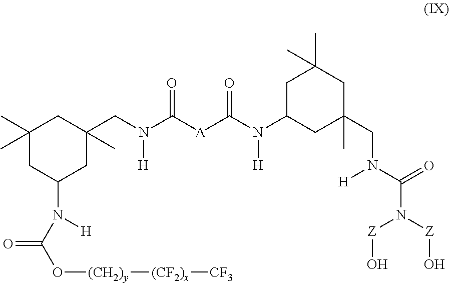
wherein
A is a building-block group of the formula —O—(CₘH₂ₘO)ₐ— wherein each m represents independently for every (CₘH₂O) unit an integer from 2 to 4 and is the same or different and a represents an integer from 2 to 30;
Z is a (CₚH₂ₚ) unit;
p is an integer from 2 to 8;
x is an integer from 0 to 9; and
y is an integer from 2 to 12.

24. A process for forming a polymerizable compound according to claim 16 comprising the steps of:
(a) reacting at least one polymeric polyol having two or more repeating units and two or more hydroxyl groups with at least one diisocyanates; then
(b) reacting the product with at least one alcohol or amine comprising a fluorinated organic moiety of 1 to 18 carbon atoms and 35 to 85 wt % of fluorine, and then
(c) reacting the product with an organic compound comprising a polymerizable building-block group.

25. A process for forming a polymerizable compound according to claim 16, comprising the steps of:
(a) reacting at least one alcohol or amine comprising a fluorinated organic moiety of 1 to 18 carbon atoms and 35 to 85 wt % of fluorine with at least one diisocyanates; and then
(b) reacting the product with at least one polymeric polyol comprising two or more repeating units and also one or more than one hydroxyl group and a polymerizable moiety.

26. A polymer comprising a monomer according to claim 16.

27. The polymer according to claim 26, wherein a polyacrylate, polymethacrylate, polyurethane or polyurea is concerned.

28. A method comprising coating a surface with the composition according to claim 26.

29. A method according to claim 28, wherein the coating is water-repellant and dirt-repellent.

30. A method according to claim 28, wherein the surface is selected from the group consisting of an aircraft wing, a roof, a doorway, a rotor and a wind mill blade.

31. The surface coated according to the method of claim 28.
